(19) United States
(12) Patent Application Publication
(10) Pub. No.: US 2026/0216726 A1
(43) Pub. Date: Jul. 30, 2026

(54) MICROFLUIDIC CHIP FOR ANALYZING CELL MIGRATION AND CELL-CELL INTERACTIONS

(71) Applicant: CHILDREN'S NATIONAL MEDICAL CENTER, Washington, DC (US)

(72) Inventor: Nitin AGRAWAL, Washington, DC (US)

(73) Assignee: CHILDREN'S NATIONAL MEDICAL CENTER, Washington, DC (US)

(21) Appl. No.: 19/143,402

(22) PCT Filed: Jan. 24, 2024
(86) PCT No.: PCT/US2024/012785

§ 371 Date: Jun. 26, 2025

Related U.S. Application Data
(60) Provisional application No. 63/481,381, filed on Jan. 24, 2023.

(51) Int. Cl. B01L 3/00

(52) U.S. Cl. CPC B01L 3/502746; B01L 3/502761; B01L 2300/0816; B01L 2300/089; B01L 2400/0487

(57) ABSTRACT

A microfluidic device including a first chamber connected to a second chamber by a network of fluid gradient channels; and a pressure-balancing channel connecting a first well to a second well, wherein the first well overlaps with a port of the first chamber and the second well overlaps with a port of the second chamber, and a fluidic resistance of the pressure-balancing channel is less than a fluidic resistance of each fluid gradient channel of the fluid gradient channels.

### CROSS-REFERENCE TO RELATED APPLICATIONS

The present application claims priority to U.S. Provisional Application No. 63/481,381, filed Jan. 24, 2023, which is incorporated herein by reference in its entirety for all purposes.

### BACKGROUND

### Field

The present disclosure is related to microfluidic devices.

### Description of the Related Art

Directional cell migration or chemotaxis is a fundamental biological process involving the movement of cells or organisms in response to chemical signals in their microenvironment. It plays a pivotal role in a myriad of physiological as well as pathological processes e.g., embryonic development, tissue repair and regeneration, and disease progression/suppression in conditions including cancer, sepsis, atherosclerosis, and arthritis.

In vivo studies are complex and obscure the monitoring of migrating cells in response to specific stimuli. Therefore, by establishing spatial chemical gradients in vitro, the influence of specific chemokines on cell migration behavior can be elucidated. Artificial chemical gradients can also facilitate scientific study in areas of extracellular vesicles, axonal biology in the central nervous system, and others where cellular co-cultures are relevant and cell functions are influenced by the gradients of secreted or recombinant molecules.

The foregoing “Background” description is for the purpose of generally presenting the context of the disclosure. Work of the inventors, to the extent it is described in this background section, as well as aspects of the description which may not otherwise qualify as prior art at the time of filing, are neither expressly or impliedly admitted as prior art against the present disclosure.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a microfluidic device, comprising: a first chamber connected to a second chamber by a network of fluid gradient channels; and a pressure-balancing channel connecting a first well to a second well, wherein the first well overlaps with a port of the first chamber and the second well overlaps with a port of the second chamber, and a fluidic resistance of the pressure-balancing channel is less than a fluidic resistance of each fluid gradient channel of the fluid gradient channels.

In one embodiment, the present disclosure is related to a microfluidic device, comprising: a gradient layer including a first chamber connected to a second chamber by a network of fluid gradient channels, each chamber having a port; and a balancing layer including a pressure-balancing channel connecting a first well to a second well, wherein the balancing layer is bonded to the gradient layer such that the first well of the balancing layer is aligned with the port of the first chamber and the second well of the balancing layer is aligned with the port of the second chamber, and a fluidic resistance of the pressure-balancing channel is less than a fluidic resistance of each fluid gradient channel of the fluid gradient channels.

In one embodiment, the present disclosure is related to a microfluidic device, comprising: gradient layer including a first chamber, a second chamber, and a third chamber, the first chamber being connected to the second chamber by a first network of fluid gradient channels, the second chamber being connected to the third chamber by a second network of fluid gradient channels, and the third chamber being connected to the first chamber by a third network of fluid gradient channels, each chamber having a port; and a balancing layer including a first pressure-balancing channel connecting a first well and a second well, a second pressure-balancing channel connecting the second well and a third well, and a third pressure-balancing channel connecting the third well and the first well, wherein the balancing layer is bonded to the gradient layer such that the first well of the balancing layer is aligned with the port of the first chamber, the second well of the balancing layer is aligned with the port of the second chamber, the third well of the balancing layer is aligned with the port of the third chamber, and a fluidic resistance of each pressure-balancing channel is less than a fluidic resistance of each fluid gradient channel of the first, second and third fluid gradient channels.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an illustration of a network of gradient channels, according to one embodiment of the present disclosure;

FIG. 2 is an illustration of a gradient layer, according to one embodiment of the present disclosure;

FIG. 3A is an exploded view of a microfluidic device, according to one embodiment of the present disclosure;

FIG. 3B is a view of an assembled microfluidic device, according to one embodiment of the present disclosure;

FIG. 4 is an illustration of a microfluidic device, according to one embodiment of the present disclosure;

FIG. 5 is a method of fabricating a microfluidic device, according to one embodiment of the present disclosure;

FIG. 6 is an illustration of an assembled microfluidic device, according to one embodiment of the present disclosure;

FIG. 7A is an illustration of fluid flow in a pressure-balancing channel, according to one embodiment of the present disclosure;

FIG. 7B is an illustration of fluid flow in a network of gradient channels, according to one embodiment of the present disclosure;

FIG. 8A is an illustration of a gradient in a microfluidic device, according to one embodiment of the present disclosure;

FIG. 8B is an illustration of a gradient in a microfluidic device, according to one embodiment of the present disclosure;

FIG. 8C is an illustration of a gradient in a microfluidic device, according to one embodiment of the present disclosure;

FIG. 9 is a graph of protein concentration in a microfluidic device, according to one embodiment of the present disclosure;

FIG. 10 is a model of fluid flow in a microfluidic device, according to one embodiment of the present disclosure;

FIG. 11 is a model of a concentration gradient in a microfluidic device, according to one embodiment of the present disclosure;

FIG. 12 is an image of a microfluidic device, according to one embodiment of the present disclosure;

FIG. 13 is an image of a microfluidic device, according to one embodiment of the present disclosure; and

FIG. 14 is a graph of cell count in a microfluidic device, according to one embodiment of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present disclosure can be embodied and viewed in many different ways.

Furthermore, as used herein, the words “a,” “an,” and the like generally carry a meaning of “one or more,” unless stated otherwise.

Numerous modifications and variations are possible in light of the above teachings. It is therefore to be understood that within the scope of the appended claims, the disclosure can be practiced otherwise than as specifically described herein.

In one embodiment, the present disclosure is directed towards a standalone microfluidic device configured to create a stable chemical concentration gradient between two or more solutions. The device can be used for chemotaxis assays in order to observe real-time cell movement in response to the chemical gradient. The device, as described herein, can be used to eliminate pressure differentials between solutions in order to create and maintain a stable, diffusion-based gradient. A diffusion-based chemical gradient can be used to mimic in vivo cell environments in in vitro experiments. For example, cells can secrete cytokines, chemokines, and exosomes, and the secreted molecules (factors) can diffuse away from the source cells over time to form a concentration gradient across an area surrounding the source cells. Other cells in the environment can respond to the gradient, e.g., by polarizing, migrating towards a certain location, activating cellular pathways, growing directionally towards certain chemical signals e.g., as in axonal growth of neurons, or performing certain cell functions as a result of exposure to the secreted molecules. Studying these cell behaviors can be helpful for understanding cell-to-cell interactions and physiological and pathological processes such as organogenesis, tissue repair and regeneration, disease progression, immune responses, axonal biology, etc. Cell migration behavior can also be used for diagnostic purposes and development of personalized therapies.

In one embodiment, the microfluidic device of the present disclosure can include at least two chambers. The two chambers can be connected by a network of channels. The channels can be referred to herein as gradient channels or migration channels. A concentration gradient between a first solution in a first chamber and a second solution in a second chamber can be established across the network of gradient channels as a result of diffusion between the two chambers. The concentration gradient across the network can be used for cell migration studies as well as studies of non-migratory cell behaviors such as growth or factor secretion. In one embodiment, the concentration gradient can reach a stable state based on the volume and/or concentration of molecules in each chamber and the dimensions of the network of gradient channels. For example, the concentration gradient of a molecule can be approximately linear across the gradient channels. In one embodiment, larger particles only experience Brownian motion within the gradient channel demonstrating zero convective flow and a perfectly balanced pressure differential across the channel, which is necessary to achieve diffusion-based gradients of secreted molecules.

In one embodiment, the network of gradient channels can include one or more gradient channels connecting a first chamber and a second chamber. The gradient channels can be approximately parallel with each other and can be separated from each other by a gap. The gradient channels can be coplanar with the first chamber and the second chamber. In one embodiment, the gradient channels can be approximately perpendicular to the walls of the first chamber and the second chamber at the location of the gradient channels.

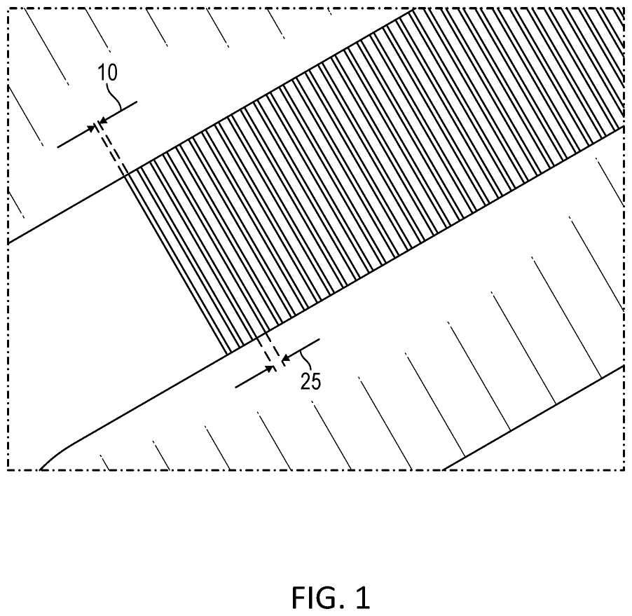

FIG. 1 is an illustration of a network of gradient channels, according to one embodiment of the present disclosure. The network can include a plurality of gradient channels. In one embodiment, the network can include 50 gradient channels. Each gradient channel can connect a body of a first chamber and a body of a second chamber. In one embodiment, each gradient channel can be approximately 10 microns (um) in width. In one embodiment, each gradient channel can be separated from an adjacent channel by a gap of approximately 25 microns (um). In one embodiment, the gradient channels can be approximately 1-10 microns (um) wide and approximately 1-10 microns (um) high (deep). As non-limiting examples, the gradient channels can be rectangular or cylindrical. In one embodiment, the dimensions of the gradient channels can be set based on an average cell size. For example, the width of the gradient channels can be set so that a type of cell (e.g., eukaryotic cell) cannot flow freely via hydrodynamic forces through the gradient channels.

In one embodiment, the microfluidic device can include more than two chambers. In one embodiment, each chamber can be connected to each of the adjacent chamber via a separate network of gradient channels. For example, in a three-chamber device, a first chamber can be connected to a second chamber via a first network, the second chamber can be connected to a third chamber via a second network, and the third chamber can be connected to the first chamber via a third network. It can be appreciated that the device described herein is not limited to a certain number of chambers or networks. The device can include a number of chambers for a number of solutions that are configured such that hydrodynamic pressures across individual chambers will be balanced by a network of balancing channels, the network providing diffusion-based gradient formation in the gradient channels across the solutions. In one embodiment, the microfluidic device can include n chambers, wherein each chamber is connected to each of the remaining n−1 chambers via a network of gradient channels. In one embodiment, a microfluidic device having n chambers can have

$$\frac{{n}^{2} - n}{2}$$

networks of gradient channels. Various chamber arrangements (e.g., chambers arranged in a polygonal shape, or in a straight line, or approximately parallel to each other) can be implemented, and the chambers can be connected using any number of networks. In this manner, a single microfluidic device can be used to create concentration gradients between a plurality of solutions.

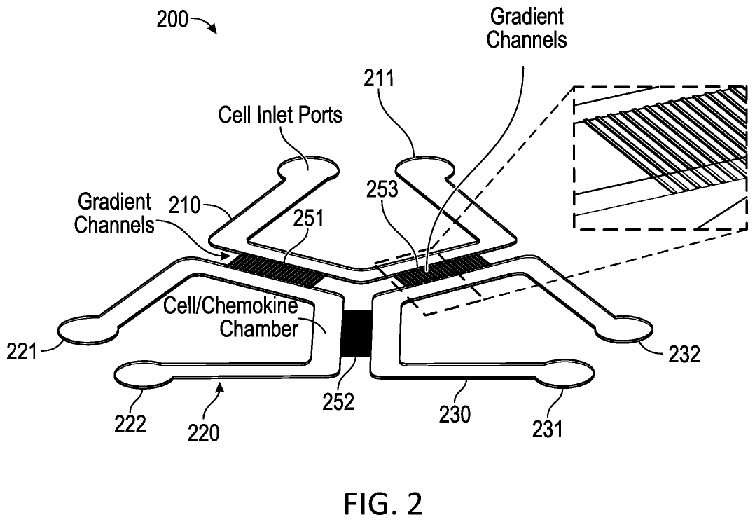

FIG. 2 is an illustration of three chambers of a microfluidic device 200, according to one embodiment of the present disclosure. Each chamber 210, 220, 230 can be an elongated chamber having a first port 211, 221, 231 and a second port 212, 222, 232. The ports can be openings (inlets) for filling the chambers with a solution, e.g., a cell solution. The ports can be located at a first end of the chamber and a second end of the chamber. In one embodiment, the chamber can include one or more bends to spatially optimize the placement of ports, as illustrated in FIG. 2. It can be appreciated that the device 200 is illustrated as a non-limiting example, and that a microfluidic device can have, at a minimum, one network of gradient channels connecting two chambers. The chambers can contain cells or cytokines. A first network of gradient channels 251 can connect a segment of a first chamber 210 to a segment of a second chamber 220. A second network of gradient channels 252 can connect a segment of the second chamber 220 to a segment of the third chamber 230. A third network of gradient channels 253 can connect a segment of the third chamber 230 to a segment of the first chamber 210. Each network can be formed by a number of parallel gradient channels. The networks of gradient channels can be distinct from each other and may not share any gradient channels. In one embodiment, the first chamber 210 can be filled with a cell/chemokine solution. The first chamber can then be a chemokine source. The chemokines can diffuse away from the first chamber 210 and towards the second chamber 220 via a network of gradient channels 251 to form a concentration gradient across the gradient channels.

In a typical microfluidic system, hydrodynamic pressure differentials between fluid reservoirs can occur over time due to random perturbations, movement of fluid, and unequal rates of evaporation. For example, a pressure differential can form between a first chamber and a connected second chamber. These pressure differentials can result in fluid flow across the gradient channels connecting the chambers in order to equalize the pressure. The pressure-driven fluid flow disturbs the diffusion-based concentration gradient in the gradient channels. Fluid movement between the chambers is then driven by convective forces arising from pressure differences diminishing the formation of diffusion-based gradients.

In one embodiment, the microfluidic device can include one or more pressure-balancing channels configured to neutralize pressure differentials between chambers while maintaining concentration gradients across networks of gradient channels. A pressure-balancing channel can connect a first chamber to a second chamber and provide an alternative route for bidirectional fluid flow between the two chambers. In one embodiment, the fluidic resistance of a pressure-balancing channel (R_{bal}) can be less than the fluidic resistance of a gradient channel (R_{grad}). As a result, fluid is more likely to flow across the pressure-balancing channel than a network of gradient channels in order to stabilize a pressure differential between chambers. Fluid in the gradient channel can thus remain stagnant.

In one embodiment, the gradient channels and the pressure-balancing channels can have rectangular cross-sections. The fluidic resistance (R) of a microfluidic channel with a rectangular cross-section can be characterized by the following equation:

$$R = \frac{1}{1 - 0.63 ( \frac{h}{w} )} \frac{1 {2}_{η} L}{{h}^{3} w}$$

Wherein h is the height of the channel, w is the width of the channel, L is the length of the channel (L>>w), and y is the dynamic viscosity of the fluid. In one embodiment, the pressure-balancing channel can be wider and/or taller (deeper) than the gradient channels. In one embodiment, the pressure-balancing channel can be longer than the gradient channels. In one example, a gradient channel can be approximately 10 microns (um) in width, 4 μm in height, and 500 microns (um) in length, and a pressure-balancing channel can be approximately 100 μm in width, 50 microns (um) in height, and 6000 microns (um or 6 mm) in length. The increase in length of the pressure-balancing channel is offset by the increase in width and height such that

$$\frac{{R}_{g r a d}}{{R}_{b a l}}$$

is approximately 1100. In one example, the pressure-balancing channel can be approximately 200 microns (um) in width, 60 microns (um) in height, and 6000 microns (um) in length, such that

$$\frac{{R}_{g r a d}}{{R}_{b a l}}$$

is approximately 5000. Therefore, it is more likely that the random pressure differentials across adjacent chambers will cause the fluid to flow through the pressure-balancing channels than the gradient channels.

In one embodiment, the gradient channels and/or the pressure-balancing channels can be cylindrical. The fluidic resistance of a cylindrical channel is also dependent on the dimensions (diameters, length) of the cylindrical channel. The dimensions of cylindrical gradient channels and/or cylindrical pressure-balancing channels can be configured in the microfluidic device such that the fluidic resistance of the pressure-balancing channel is similarly less than the fluidic resistance of the gradient challenges.

In one embodiment, a pressure-balancing channel can connect a port or ports of a first chamber and a port or ports of a second chamber. The ports can be openings whereby fluid can flow into or out of a chamber. Fluid can then flow directly between the ports to balance pressure differentials in the chambers. In one embodiment, a microfluidic device can include more than one pressure-balancing channel. For example, a microfluidic device having three chambers can include three pressure-balancing channels. A first pressure-balancing channel can connect ports of a first chamber to ports of a second chamber, a second pressure-balancing channel can connect the ports of the second chamber to ports of a third chamber, and a third pressure-balancing channel can connect the ports of the third chamber to the ports of the first chamber. In one embodiment, the pressure-balancing channels can form a continuous channel, e.g., a loop. In one embodiment, a pressure-balancing channel can further connect ports of a same channel to allow fluid to flow to and from either port. In one embodiment, the pressure-balancing channels can be co-planar with the gradient channels or they can be fabricated in separate layers at different planes. Regardless of their planar arrangement, the balancing channels can connect the adjacent chambers to allow neutralization of pressure differentials.

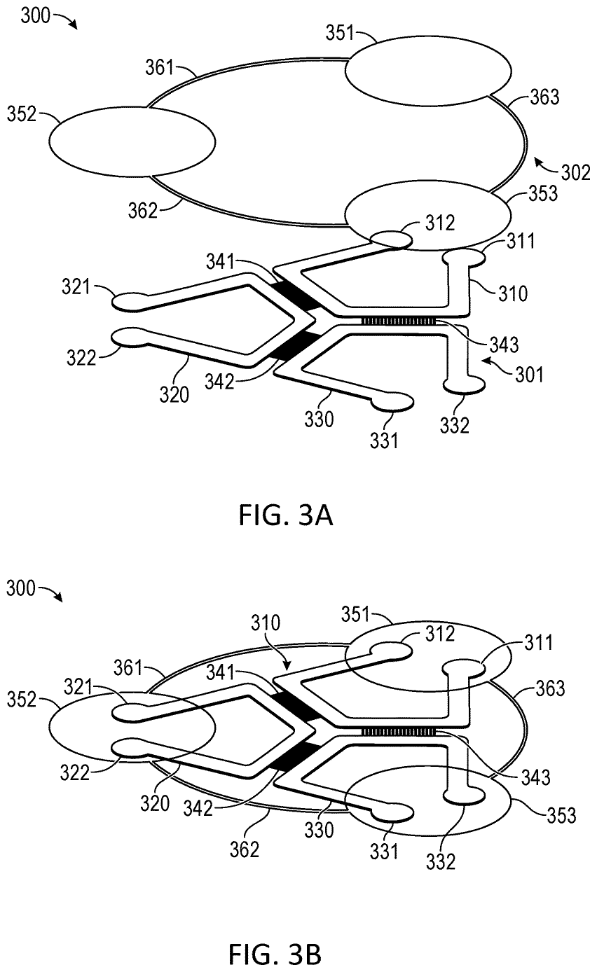

FIG. 3A is an illustration of a first layer 301 and a second layer 302 of a microfluidic device 300 according to one embodiment. The first layer (gradient layer) 301 can form the chambers and gradient channels. For example, the gradient layer 301 can form a first chamber 310, a second chamber 320, and a third chamber 330. Each chamber can include a first port 311, 321, 331 and a second port 312, 322, 332. The chambers can be connected via a first network of gradient channels 341, a second network of gradient channels 342, and a third network of gradient channels 343.

The second layer (balancing layer) 302 can form the pressure-balancing channels. In one embodiment, a pressure-balancing channel can be formed between a well or reservoir at each end of the pressure-balancing channel. The wells can form openings to the pressure-balancing channel. For example, the balancing layer 302 can form a first well 351, a second well 352, and a third well 353. A first pressure-balancing channel 361 can connect the first well 351 to the second well 352. A second pressure-balancing channel 362 can connect the second well 352 to the third well 353. A third pressure-balancing channel 363 can connect the third well 353 to the first well 351. The pressure-balancing channels 361, 362, 363 and the wells 351, 352, 353 can form a continuous feature. For example, the channels and wells can form a continuous ring, as illustrated in FIG. 3A.

In one embodiment, each layer can be a polymer layer (e.g., a polydimethylsiloxane (PDMS) layer or any other cell-compatible material such as glass, polystyrene, cyclic olefin copolymer etc.). The features of a layer (chambers, channels, wells, etc.) can be patterned into a surface of the layer. In one embodiment, the chambers and networks of gradient channels of the gradient layer can be formed at the bottom surface of the gradient layer. The ports can extend through the gradient layer from the bottom surface to the opposite (top) surface of the gradient layer. In one embodiment, the wells and pressure-balancing channels of the balancing layer can be formed at a bottom surface of the balancing layer or formed co-planar with the gradient layer. The bottom surface of the balancing layer can be in contact with the top surface of the gradient layer when the device is assembled. In one embodiment, the bottom layer (e.g., the gradient layer) can be bonded to a glass slide or another layer of a transparent substrate to seal the features at the bottom surface of the bottom layer. The top surface of the gradient layer and the bottom surface of the balancing layer can be bonded to each other to seal the features at the adjacent surfaces while allowing fluid movement between the layers.

FIG. 3B is an illustration of the microfluidic device 300 wherein the gradient layer is bonded to the balancing layer. In one embodiment, the wells can be aligned with ports of a chamber such that fluid can flow from a port of a chamber into the well and through the pressure-balancing channel connected to the well. The layers can be aligned such that each well of the balancing layer 302 overlaps with the two ports of a chamber. For example, the first well 351 overlaps with ports 311, 312 of the first chamber 310, the second well 352 overlaps with ports 321, 322 of the second chamber 320, and the third well 353 overlaps with ports 331, 332 of the third chamber 330. Fluid from a chamber in the gradient layer can flow through a port and into an overlapping well in the balancing layer. In this manner, fluid can flow from the first chamber 310 to the second chamber 320 in the balancing layer via the first pressure-balancing channel 361 connecting the first well 351 to the second well 352. Fluid can flow from the second chamber 320 to the third chamber 330 in the balancing layer via the second pressure-balancing channel 362 connecting the second well 352 to the third well 353. Fluid can flow from the third chamber 330 to the first chamber 310 in the balancing layer via the third pressure-balancing channel 363 connecting the third well 353 to the first well 351. The channels of the balancing layer 302 provide an alternative route of lower fluidic resistance for fluid to flow between the chambers of the gradient layer. Fluid flow in the balancing layer can stabilize the pressure between the chambers while fluid in the gradient layer (e.g., in the gradient channels) is stable. In this manner, the fluid in the gradient channels can be driven by diffusion and is not subject to convective flows.

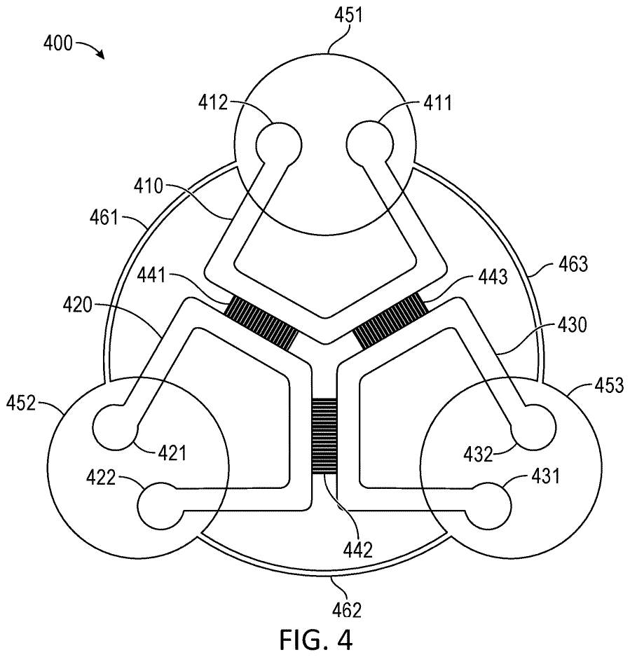

FIG. 4 is an overhead view of a microfluidic device 400 according to one embodiment. The microfluidic device 400 can include three chambers 410, 420, 430. The chambers can be connected to each other via a first network of gradient channels 441, a second network of gradient channels 442, and a third network of gradient channels 443. A first well 451 can encompass or overlap with a first port 411 and second port 412 of the first chamber 410. A second well 452 can encompass or overlap with a first port 421 and second port 422 of the second chamber 420. A third well 453 can encompass or overlap with a first port 431 and second port 432 of the third chamber 433. A first pressure-balancing channel 461 can connect the first well 451 to the second well 452; a second pressure-balancing channel 462 can connect the second well 452 to the third well 453; and a third pressure-balancing channel 463 can connect the third well 453 to the first well 451. In one embodiment, the pressure-balancing channels do not overlap or intersect with the gradient channels in the plane illustrated in FIG. 4.

The features of the microfluidic device 400 can be formed in more than one layer (e.g., the gradient layer and balancing layer described with reference to FIGS. 3A and 3B) or can be formed in a single layer. In one embodiment, the chambers 410, 420, 430 of the microfluidic device 400 can be approximately 500 μm in width. The ports 411, 421, 431 and 412, 422, 432 of each chamber can be approximately circular ports and can each have a radius of approximately 500 microns (um). The networks of gradient channels 441, 442, 443 can be approximately 500 microns (um) in length. The gradient channel length can result in a concentration gradient slope that mimics an in vivo cell environment. In addition, the gradient channel length can be a representative model of the distance that a cell may travel via chemotaxis in the body. In one embodiment, the wells 451, 452, 453 can each have a radius of approximately 2000 μm. The wells can be larger than at least one port 411, 421, 431, 412, 422, 432 of a chamber in order to encompass the entirety of at least one port. The pressure-balancing channels 461, 462, 463 can be approximately 100 microns (um) in width.

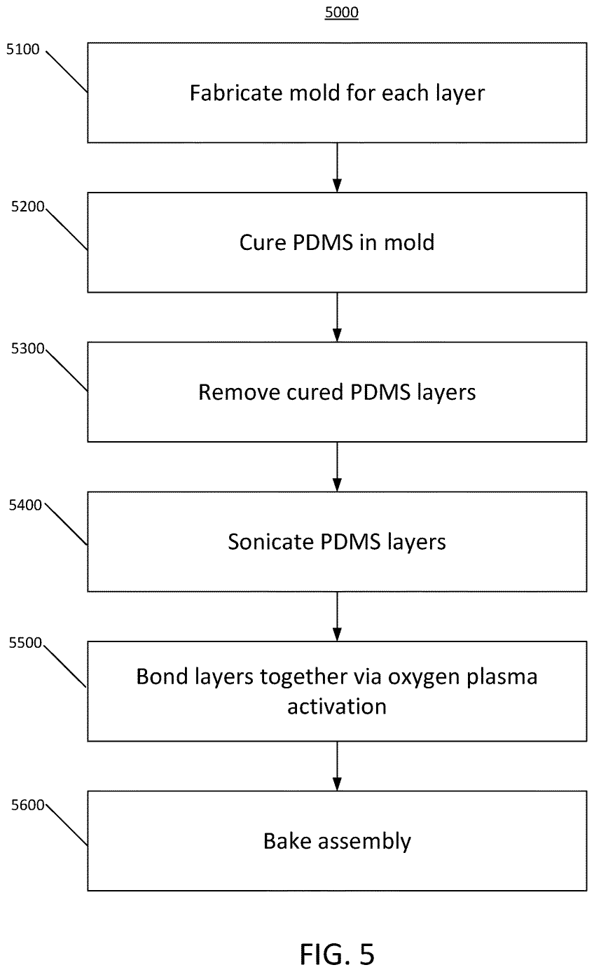

FIG. 5 is a method 5000 of fabricating a microfluidic device, according to one embodiment of the present disclosure. The microfluidic device can include a first layer (gradient layer) and a second layer (balancing layer) or can be formed by a single layer. In step 5100, a mold can be fabricated for each layer of the microfluidic device. The mold can be formed from a silicon wafer. The features of the layer can be prepared via computer-aided design (CAD) and can be formed in the silicon wafer via a soft lithography process (e.g., direct-write lithography). Alternatively or additionally, the features can be formed using techniques such as embossing, injection molding, 3D printing, or micromilling. In step 5200, the layer can be fabricated by curing a polymer in the mold. In one embodiment, the polymer can be an elastomer such as PDMS. The PDMS can be mixed with a curing agent in a ratio of 10 to 1 (PDMS to curing agent). In one embodiment, a gradient layer can be approximately 1-2 millimeters (mm) in thickness, and a balancing layer can be approximately 4-5 mm in thickness. The PDMS can be left to cure overnight at 65° C.

In step 5300, the cured layer can be removed from the mold. Excess PDMS surrounding the features can be removed when needed. The ports and wells can be punched into the PDMS, e.g., with a needle puncher or a leather hole punch. In step 5400, the layers can be sonicated in a 25% isopropanol water solution to remove debris and can be dried for at least one hour at 65° C. In step 5500, the layers can be bonded together. A first PDMS layer can be bonded to a glass slide (e.g., 1 inch by 3 inch glass slide) via known oxygen plasma activation methods. In one embodiment, the first PDMS layer can be placed in contact with the glass slide for approximately one minute after plasma activation for bonding. In an embodiment wherein the microfluidic device includes two PDMS layers, the second PDMS layer (top layer, balancing layer) can be bonded to the first PDMS layer (bottom layer, gradient layer) via a oxygen plasma activation method. The bottom surface of the top layer can be bonded to the top surface of the bottom layer such that the wells and ports are aligned as described with reference to FIG. 4. In step 5600, the bonded assembly can be heated (baked) at 95° C. for 15 minutes to ensure strong covalent bonding between each layer.

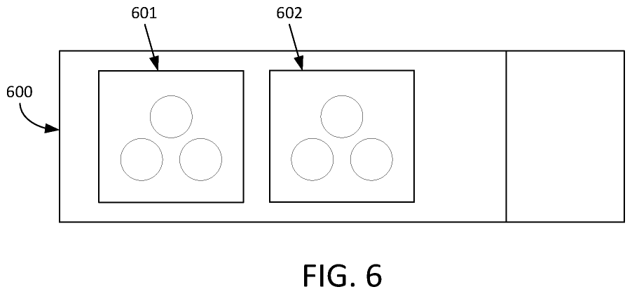

FIG. 6 is an illustration of a first fabricated microfluidic device 601 and a second fabricated microfluidic device 602. Each microfluidic device can be fabricated according to the method of FIG. 5. The microfluidic devices 601 and 602 can both be bonded to a single glass slide 600.

In one embodiment, the microfluidic device can be exposed to oxygen plasma for approximately 30 seconds prior to usage. Exposure to oxygen plasma can cause the channels of the device to become hydrophilic in order to facilitate loading of solutions into the channels and chambers. Alternatively or additionally, the microfluidic device can be coated with a hydrophilic coating such as polylysine at a 20 μg/mL concentration during or after the fabrication process. In one embodiment, a first volume of a solution can first be loaded into an inlet port of each chamber. The first volume can be approximately 2 μL. The first volume can spread through the chambers via capillary action. A second volume of each respective solution can then be introduced into the respective wells or reservoirs. The second volume can be greater than the first volume, e.g., approximately 40 μL. gradient layer.

In one embodiment, a different solution can be loaded into each chamber. In one embodiment, the solutions can be cell solutions and/or chemokine solutions. For example, the solution can be a single-cell suspension. The cells can be stained for imaging and tracking. In one example, a chemokine solution can be prepared in a cell medium or phosphate-buffered saline (PBS). In one embodiment, the chambers can be loaded in an order in order to prevent early diffusion of solutions during the loading. For example, a lower chamber can be loaded prior to an upper chamber.

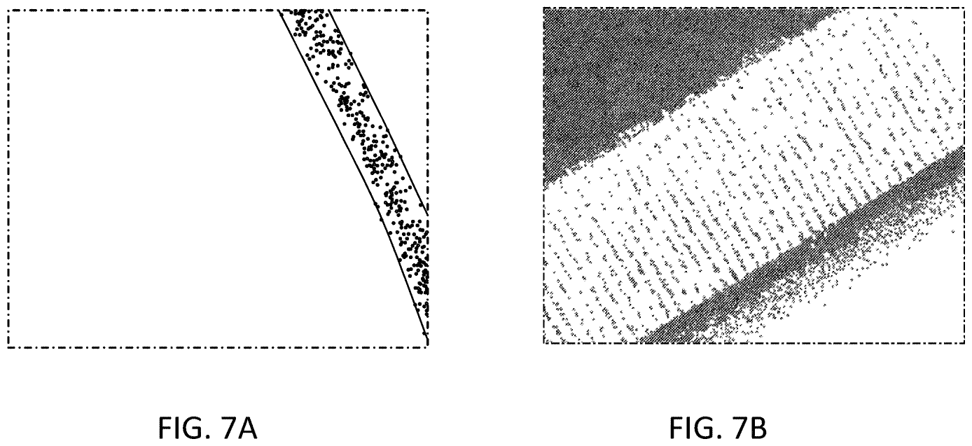

FIG. 7A is an image of fluid flow through a pressure-balancing channel of a microfluidic device. The sample fluid is a suspension of fluorescent beads of a 0.5 μm diameter. The sample fluid is introduced into each well of the microfluidic device. The sample fluid moves between the wells via the pressure-balancing channels. In one embodiment, the fluid flow through the pressure-balancing channels can decrease over time after the device is loaded as the pressure of each well stabilizes. In one embodiment, the fluid flow through the pressure-balancing channels can remain non-zero to continuously balance any pressure differentials.

FIG. 7B is an image of fluid in a network of gradient channels connecting chambers in the microfluidic device. The fluid can be stable in the gradient channels such that the fluorescent beads are stagnant in the gradient channels. In one embodiment, the fluid in the gradient channels and surrounding segments of the chambers can be only subject to Brownian motion and does not exhibit convective flow even after several hours of incubation.

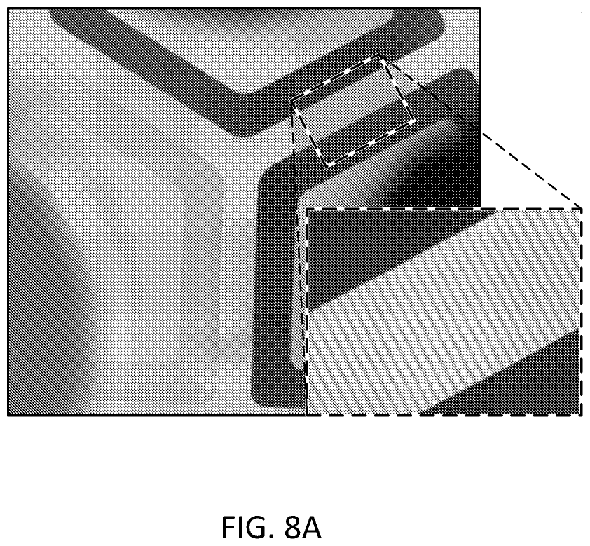

FIG. 8A is an image of a microfluidic device loaded with water-soluble colored dye. Each chamber can be loaded with a different colored dye. A gradient between each chamber can be formed in the network of gradient channels, as evidenced by the color change across each network. In one embodiment, the gradient can be a linear gradient. In one embodiment, fluid flow between the chambers through the pressure-balancing channels (not pictured) can be minimal such that the bulk concentration of each chamber and the network of gradient channels is not changed. FIG. 8B is an image of a single network of gradient channels of the device of FIG. 8A on day 1 after loading. The gradient between the yellow chamber and the blue chamber can be formed across the gradient channels. FIG. 8C is an image of the network of FIG. 8B on day 7 after loading. On day 7, fluid exchange can occur between the chambers through the balancing channel or via diffusion, as evidenced by the color change of the two chambers. However, the gradient between the chambers across the gradient channels can still be maintained. In one embodiment, the fluid flow between chambers over time can be useful for inducing cell-to-cell communication that would normally occur in vivo as cell secretion factors transport from a chamber to the adjacent over time. The microfluidic device can be used to induce post-migration cell responses to an established gradient for observation over time.

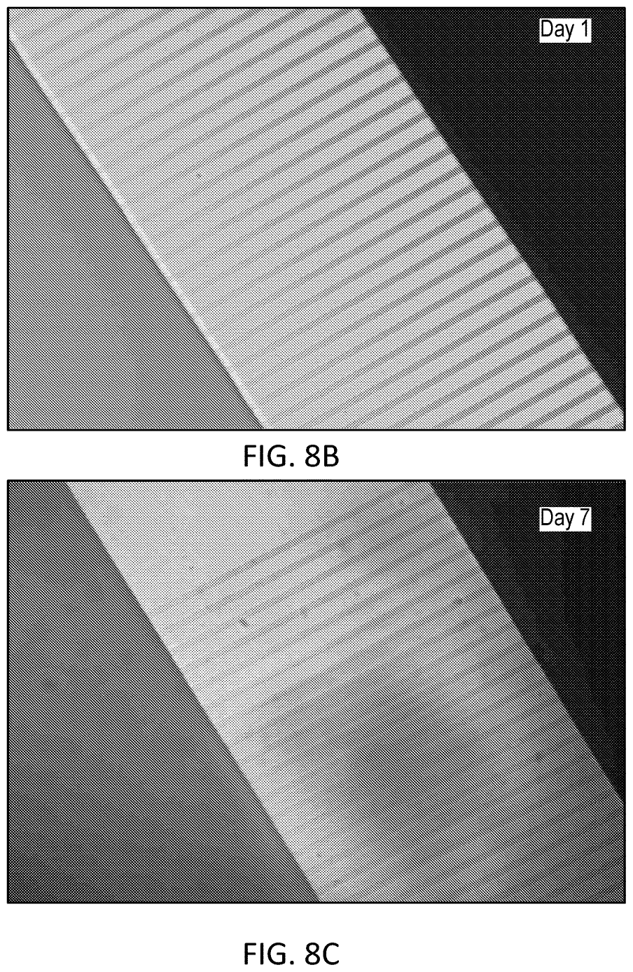

FIG. 9 is a graph of the concentration change of the bulk reservoir fluid over time as a result of diffusion and fluid transport through the balancing channel. Chemokine and cell-secreted cytokine solutions used for chemotaxis assays typically have concentrations in the range of 8-10 picomolar or 50-100 μg/mL, while recombinant chemoattractant concentrations used in migration studies are typically less than 50 ng/mL. These concentrations are significantly lower than the concentrations of colored dye used to obtain the images of FIGS. 8A-8C. Therefore, it is useful to determine whether fluid flow between reservoir wells affects chamber concentration for less-concentrated biomolecule solutions. In one embodiment, a first well can be loaded with a 25 mg/mL bovine serum albumin (BSA) solution. An adjacent well can be loaded with pure water. A volume of solution (e.g., 2 μL) can be sampled from each well to determine the protein concentrations in the wells. The wells can be replenished with the same volume that is removed to maintain the hydrodynamic pressure. In one embodiment, the microfluidic device can be incubated in a CO₂ incubator during usage.

The graph of FIG. 9 indicates that the BSA concentration in the adjacent wells is approximately stable over a period of five days. Any fluid flow between the chambers or wells does not significantly affect the concentration of each chamber and well. Thus, the gradient formed between the chambers is also not affected. In a cell study, cells can be seeded in the segments of the chambers near the gradient channels. The concentration of secreted factors can be highest in the areas surrounding the cell and lowest within the reservoir wells, and therefore can be effective for establishing a gradient across the gradient channels.

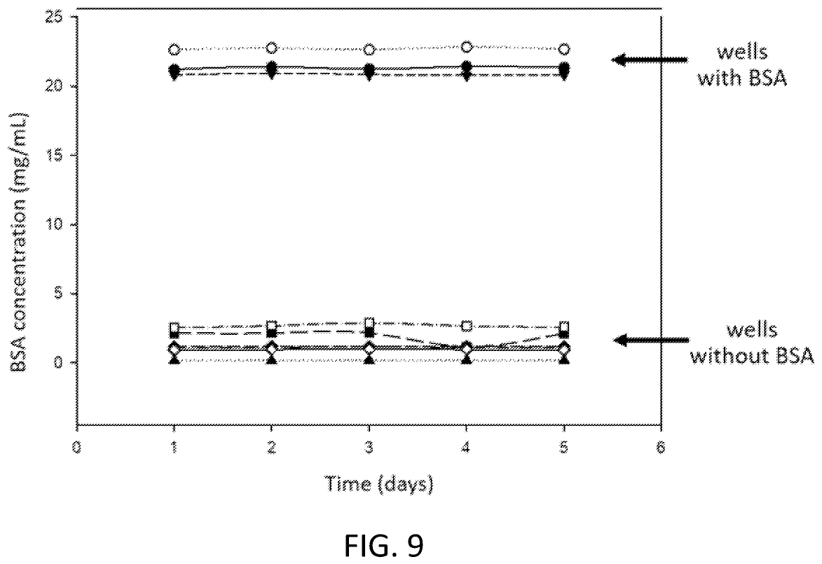

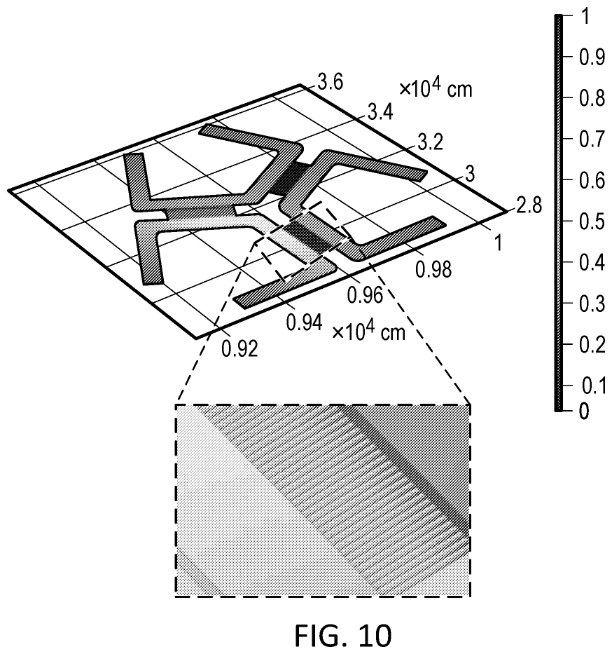

FIG. 10 is a computational model of gradient formation in a microfluidic device. The fluid flow in the device can be modeled using finite element modeling. The concentration gradient can be established across the network of gradient channels, as indicated by the color difference across the network. FIG. 11 is a graph of simulated concentration values across the length of a gradient channel. In one embodiment, the gradient can be linear when the fluid is at steady state. In one embodiment, the gradient can be non-linear. The slope and linearity of the gradient can change when cell-secreted factors are present.

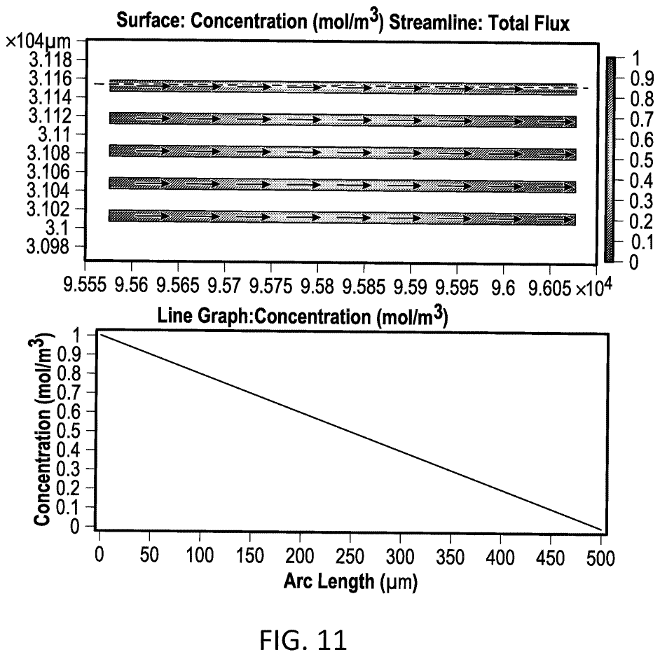

FIG. 12 is an image of the microfluidic device being used for analysis of cell migration in response to cell-secreted factors. The microfluidic device can be used effectively for simultaneous migration and cell-to-cell communication studies. For example, rhabdomyosarcoma cancer cell line RD-CCL-136 (green, top chamber) can be loaded into the first well, T-cells (red, left chamber) can be loaded into the second well, and human primary monocytes (blue, right chamber) can be loaded into the third well. The microfluidic device can be incubated with the solution for 12 hours. The T-cells can migrate towards the cancer cells as a result of the factors secreted by the cancer cells. Mutual (bidirectional) migration between the cancer cells and the monocytes can also occur. The migrations indicate that the presence of other cells influences cell motility behavior. The T-cells and monocytes do not migrate towards each other, suggesting that these two cell types do not influence each other via cell-to-cell communication.

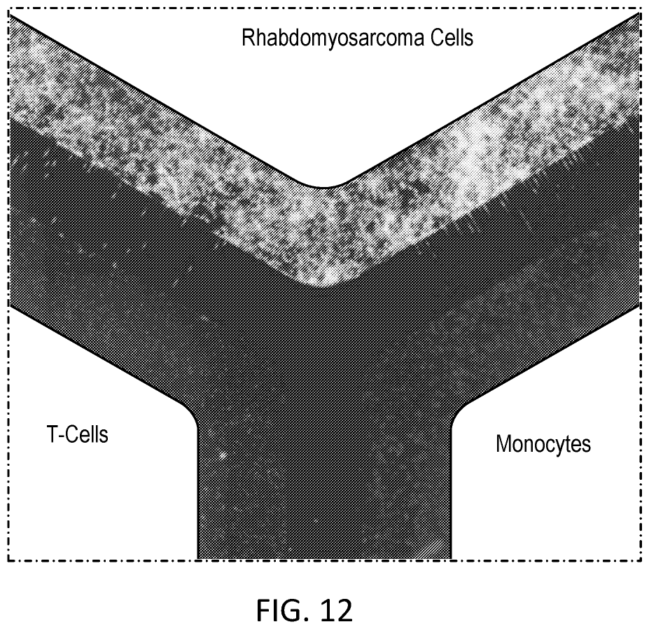

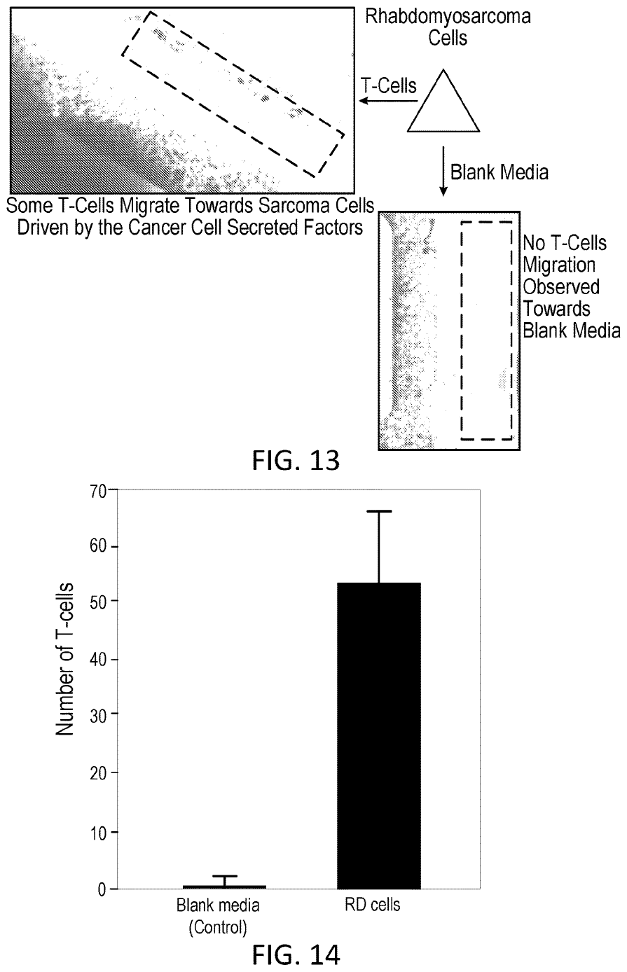

FIG. 13 is an image of the microfluidic device being used for analysis of cell migration using a negative control. RD-CCL-136 cells can be loaded into the first well, T-cells (blue) can be loaded into the second well, and blank media can be loaded into the third well. The T-cells can migrate towards the RD-CCL-136 chamber and not towards the blank media chamber. FIG. 13 is a graph of T-cells observed in the blank media chamber and the RD-CCL-136 (RD) chamber after a period of time. The negative control results can be used to confirm the effect of the cancer cells on the T-cells. The T-cells can migrate across the gradient channels towards the cancer cells in response to factors secreted by the cancer cells. However, the T-cells do not migrate towards blank media because there are no chemical factors that would induce a cell response.

The microfluidic device described herein can enable real-time observation of cell behavior at a single-cell level in an environment that reflects in vivo conditions. The real-time observation of cell behavior can result in qualitative data such as migration velocity profiles and patterns, which can be useful for determining cellular responsiveness under specific conditions. Single-cell analysis can be used to observe cellular heterogeneity and immune cells, as migrating and non-migrating cells can have different cytotoxic potentials. The microfluidic device can be used to establish a purely diffusion-based concentration gradient via the gradient channels across a longer distance than typical transwell assays. As a result, cellular migration behavior over a longer distance and/or period of time can be analyzed. The microfluidic device can also establish more than one concentration gradient between different solutions, enabling multiplex analysis. Furthermore, the horizontal orientation of the gradient channelscan normalize the effect of gravity on cells, typically associated with vertically stacked transwell chambers.

The microfluidic device can be easily fabricated as a disposable device. The microfluidic device can be used as a standalone platform without attachment to external fluidics machinery such as pumps or valves. In one embodiment, the microfluidic device can be loaded with solutions in a single initial process, and the concentration gradient can be established and sustained without additional input or fluid manipulation. The solutions can be prepared and loaded in set volumes based on the dimensions of the device features. The microfluidic device can easily be used to establish a stable chemical environment for inducing and observing cell behaviors over a period of time. Multiple microfluidic devices can be run in parallel due to the ease of use of the device. For example, a single glass slide can be bonded to multiple microfluidic devices. The microfluidic device can also be observed and imaged using common laboratory instruments such as light microscopes.

While this specification contains many specific implementation details, these should not be construed as limitations on the scope of what may be claimed, but rather as descriptions of features and applications that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

In the preceding description, specific details have been set forth, such as a particular geometry of a processing system and descriptions of various components and processes used therein. It should be understood, however, that techniques herein may be practiced in other embodiments that depart from these specific details, and that such details are for purposes of explanation and not limitation. Embodiments disclosed herein have been described with reference to the accompanying drawings. Similarly, for purposes of explanation, specific numbers, materials, and configurations have been set forth in order to provide a thorough understanding. Nevertheless, embodiments may be practiced without such specific details. Components having substantially the same functional constructions are denoted by like reference characters, and thus any redundant descriptions may be omitted.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

Obviously, numerous modifications and variations of the present disclosure are possible in light of the above teachings. It is therefore to be understood that within the scope of the appended claims, the disclosure may be practiced otherwise than as specifically described herein.

### Embodiments of the Present Disclosure May Also be as Set Forth in the Following Parentheticals:

(1) A microfluidic device, comprising: a first chamber connected to a second chamber by a network of fluid gradient channels; and a pressure-balancing channel connecting a first well to a second well, wherein the first well overlaps with a port of the first chamber and the second well overlaps with a port of the second chamber, and a fluidic resistance of the pressure-balancing channel is less than a fluidic resistance of each fluid gradient channel of the fluid gradient channels.
(2) The microfluidic device of (1), wherein the pressure-balancing channel is not intersecting with the network of fluid gradient channels.
(3) The microfluidic device of (1) to (2), wherein a width of the pressure-balancing channel is greater than a width of each fluid gradient channel of the fluid gradient channels and a height of the pressure-balancing channel is greater than a height of each fluid gradient channel of the fluid gradient channels.
(4) The microfluidic device of (1) to (3), wherein the fluid gradient channels are parallel with each other.
(5) The microfluidic device of (1) to (4), wherein a length of each fluid gradient channel of the fluid gradient channels is approximately 500 microns.
(6) The microfluidic device of (1) to (5), wherein the first and second chambers and the fluid gradient channels are formed in polydimethylsiloxane (PDMS).
(7) The microfluidic device of (1) to (6), further comprising a third chamber connected to the second chamber by a second network of second fluid gradient channels and connected to the first chamber by a third network of third fluid gradient channels, a second pressure-balancing channel connecting the second well to a third well, and a third pressure-balancing channel connecting the third well to the first well, the third well overlapping with a port of the third chamber.
(8) A microfluidic device, comprising: a gradient layer including a first chamber connected to a second chamber by a network of fluid gradient channels, each chamber having a port; and a balancing layer including a pressure-balancing channel connecting a first well to a second well, wherein the balancing layer is bonded to the gradient layer such that the first well of the balancing layer is aligned with the port of the first chamber and the second well of the balancing layer is aligned with the port of the second chamber, and a fluidic resistance of the pressure-balancing channel is less than a fluidic resistance of each fluid gradient channel of the fluid gradient channels.
(9) The microfluidic device of (8), wherein each chamber has a first port and a second port and each well is aligned with the first port and the second port of a chamber.
(10) The microfluidic device of (8) to (9), wherein a width of the pressure-balancing channel is greater than a width of each fluid gradient channel of the fluid gradient channels and a height of the pressure-balancing channel is greater than a height of each fluid gradient channel of the fluid gradient channels.
(11) The microfluidic device of (8) to (10), wherein the fluid gradient channels are parallel with each other.
(12) The microfluidic device of (8) to (11), wherein a length of each fluid gradient channel of the fluid gradient channels is approximately 500 microns.
(13) The microfluidic device of (8) to (12), wherein the gradient layer and the balancing layer are polydimethylsiloxane (PDMS) layers.
(14) The microfluidic device of (8) to (13), further comprising a third chamber connected to the second chamber by a second network of second fluid gradient channels and connected to the third chamber by a third network of third fluid gradient channels in the gradient layer, and a second pressure-balancing channel connecting the second well to a third well and a third pressure-balancing channel connecting the third well to the first well in the balancing layer, wherein the third well is aligned with the port of the third chamber.
(15) A microfluidic device, comprising: a gradient layer including a first chamber, a second chamber, and a third chamber, the first chamber being connected to the second chamber by a first network of first fluid gradient channels, the second chamber being connected to the third chamber by a second network of second fluid gradient channels, and the third chamber being connected to the first chamber by a third network of third fluid gradient channels, each chamber having a port; and a balancing layer including a first pressure-balancing channel connecting a first well and a second well, a second pressure-balancing channel connecting the second well and a third well, and a third pressure-balancing channel connecting the third well and the first well, wherein the balancing layer is bonded to the gradient layer such that the first well of the balancing layer is aligned with the port of the first chamber, the second well of the balancing layer is aligned with the port of the second chamber, the third well of the balancing layer is aligned with the port of the third chamber, and a fluidic resistance of each pressure-balancing channel of the first, second and third pressure-balancing channels is less than a fluidic resistance of each fluid gradient channel of the first, second and third fluid gradient channels.
(16) The microfluidic device of (15), wherein each chamber has a first port and a second port and each well is aligned with the first port and the second port of a chamber.
(17) The microfluidic device of (15) to (16), wherein a width of each pressure-balancing channel of the first, second and third pressure-balancing channels is greater than a width of each fluid gradient channel of the first, second and third fluid gradient channels and a height of each pressure-balancing channel of the first, second and third pressure-balancing channels is greater than a height of each fluid gradient channel of the first, second and third fluid gradient channels.
(18) The microfluidic device of (15) to (17), wherein the first fluid gradient channels are parallel with each other, the second fluid gradient channels are parallel with each other, and the third fluid gradient channels are parallel with each other.
(19) The microfluidic device of (15) to (18), wherein a length of each fluid gradient channel of the first, second and third fluid gradient channels is approximately 500 microns.
(20) The microfluidic device of (15) to (19), wherein the gradient layer and the balancing layer are polydimethylsiloxane (PDMS) layers.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A microfluidic device, comprising:
a first chamber connected to a second chamber by a network of fluid gradient channels; and
a pressure-balancing channel connecting a first well to a second well, wherein
the first well overlaps with a port of the first chamber and the second well overlaps with a port of the second chamber, and
a fluidic resistance of the pressure-balancing channel is less than a fluidic resistance of each fluid gradient channel of the fluid gradient channels.

2. The microfluidic device of claim 1, wherein the pressure-balancing channel is not intersecting with the network of fluid gradient channels.

3. The microfluidic device of claim 1, wherein a width of the pressure-balancing channel is greater than a width of each fluid gradient channel of the fluid gradient channels and a height of the pressure-balancing channel is greater than a height of each fluid gradient channel of the fluid gradient channels.

4. The microfluidic device of claim 1, wherein the fluid gradient channels are parallel with each other.

5. The microfluidic device of claim 1, wherein a length of each fluid gradient channel of the fluid gradient channels is approximately 500 microns.

6. The microfluidic device of claim 1, wherein the first and second chambers and the fluid gradient channels are formed in polydimethylsiloxane (PDMS).

7. The microfluidic device of claim 1, further comprising a third chamber connected to the second chamber by a second network of second fluid gradient channels and connected to the first chamber by a third network of third fluid gradient channels, a second pressure-balancing channel connecting the second well to a third well, and a third pressure-balancing channel connecting the third well to the first well, the third well overlapping with a port of the third chamber.

8. A microfluidic device, comprising:
a gradient layer including a first chamber connected to a second chamber by a network of fluid gradient channels, each chamber having a port; and
a balancing layer including a pressure-balancing channel connecting a first well to a second well, wherein
the balancing layer is bonded to the gradient layer such that the first well of the balancing layer is aligned with the port of the first chamber and the second well of the balancing layer is aligned with the port of the second chamber, and
a fluidic resistance of the pressure-balancing channel is less than a fluidic resistance of each fluid gradient channel of the fluid gradient channels.

9. The microfluidic device of claim 8, wherein each chamber has a first port and a second port and each well is aligned with the first port and the second port of a chamber.

10. The microfluidic device of claim 8, wherein a width of the pressure-balancing channel is greater than a width of each fluid gradient channel of the fluid gradient channels and a height of the pressure-balancing channel is greater than a height of each fluid gradient channel of the fluid gradient channels.

11. The microfluidic device of claim 8, wherein the fluid gradient channels are parallel with each other.

12. The microfluidic device of claim 8, wherein a length of each fluid gradient channel of the fluid gradient channels is approximately 500 microns.

13. The microfluidic device of claim 8, wherein the gradient layer and the balancing layer are polydimethylsiloxane (PDMS) layers.

14. The microfluidic device of claim 8, further comprising a third chamber connected to the second chamber by a second network of second fluid gradient channels and connected to the third chamber by a third network of third fluid gradient channels in the gradient layer, and a second pressure-balancing channel connecting the second well to a third well and a third pressure-balancing channel connecting the third well to the first well in the balancing layer, wherein the third well is aligned with the port of the third chamber.

15. A microfluidic device, comprising:
a gradient layer including a first chamber, a second chamber, and a third chamber, the first chamber being connected to the second chamber by a first network of first fluid gradient channels, the second chamber being connected to the third chamber by a second network of second fluid gradient channels, and the third chamber being connected to the first chamber by a third network of third fluid gradient channels, each chamber having a port; and
a balancing layer including a first pressure-balancing channel connecting a first well and a second well, a second pressure-balancing channel connecting the second well and a third well, and a third pressure-balancing channel connecting the third well and the first well, wherein
the balancing layer is bonded to the gradient layer such that the first well of the balancing layer is aligned with the port of the first chamber, the second well of the balancing layer is aligned with the port of the second chamber, the third well of the balancing layer is aligned with the port of the third chamber, and
a fluidic resistance of each pressure-balancing channel of the first, second and third pressure-balancing channels is less than a fluidic resistance of each fluid gradient channel of the first, second and third fluid gradient channels.

16. The microfluidic device of claim 15, wherein each chamber has a first port and a second port and each well is aligned with the first port and the second port of a chamber.

17. The microfluidic device of claim 15, wherein a width of each pressure-balancing channel of the first, second and third pressure-balancing channels is greater than a width of each fluid gradient channel of the first, second and third fluid gradient channels and a height of each pressure-balancing channel of the first, second and third pressure-balancing channels is greater than a height of each fluid gradient channel of the first, second and third fluid gradient channels.

18. The microfluidic device of claim 15, wherein the first fluid gradient channels are parallel with each other, the second fluid gradient channels are parallel with each other, and the third fluid gradient channels are parallel with each other.

19. The microfluidic device of claim 15, wherein a length of each fluid gradient channel of the first, second and third fluid gradient channels is approximately 500 microns.

20. The microfluidic device of claim 15, wherein the gradient layer and the balancing layer are polydimethylsiloxane (PDMS) layers.
